(19) United States
(12) Patent Application Publication
(10) Pub. No.: US 2012/0230927 A1
(43) Pub. Date: Sep. 13, 2012

(54) CYCLOMETHICONE-FREE COSMETIC AND PERSONAL CARE PRODUCTS

(75) Inventors: Kevin Gallagher, Lincroft, NJ (US); Robert Comber, Doylestown, PA (US); Abel G. Pereira, Bridgewater, NJ (US)

(73) Assignee: CRODA, INC., Edison, NJ (US)

(21) Appl. No.: 13/395,156

(22) PCT Filed: Aug. 3, 2010
(86) PCT No.: PCT/US10/44226

§ 371 Date: May 8, 2012

Related U.S. Application Data
(60) Provisional application No. 61/241,145, filed on Sep. 10, 2009.

(51) Int. Cl. A61K 8/37; A61Q 15/00; A61Q 17/04

(52) U.S. Cl. 424/59; 514/785; 424/65

(57) ABSTRACT

A personal care or cosmetic composition that includes from about 0.1% to about 99% of at least one ester of alkoxylated aromatic alcohol and fatty carboxylic acid, and from about 0.1% to 60% of at least one functional ingredient is provided. The functional ingredients in the composition may include active ingredients and additional ingredients. Especially suitable active ingredients are sunscreen active ingredients and antiperspirant active ingredients. These compositions include a reduced amount of a cyclomethicone and preferably one substantially cyclomethicone-free.

### CROSS-REFERENCE TO RELATED APPLICATIONS

This application claims the benefit of the filing date of U.S. Provisional Patent Application No. 61/241,145 filed Sep. 10, 2009, the disclosure of which is hereby incorporated herein by reference.

### BACKGROUND OF THE INVENTION

Formulation of personal care and cosmetic products, such as shampoos, conditioners, sunscreen lotions, lipstick products, mascara, and the like, presents a number of challenges. The feel and look of such products should appeal to consumers. For example, it may be desirable for certain of such products to have a non-greasy skin feel, good skin-spread ability, pleasant smell, and so on.

Personal care and cosmetic products often include chemical compounds that function as emollients. The emollients contribute to the desired skin feel, but often serve other purposes in the formulation as well. Emollients can significantly influence the dissolution/dispersion of the functional ingredients in the formulation. For example, the sunscreen products include sunscreen active ingredients that are preferably uniformly dissolved or dispersed in the formulation. However, certain commonly used sunscreen active ingredients such as pigments or other particulates exhibit poor solubility/dispersibility/wetability in formulations. Any non-uniform distribution of the active ingredient may lead to a reduction in the quality and performance of the sunscreen product. Therefore, the emollient component of the product formulation should not negatively effect the solubility of the functional ingredient(s), and, preferably, should help solubilize or disperse the functional ingredient(s) in the formulation.

Certain aromatic esters are presently used as ingredients in the personal care and cosmetic products. For example, Croda, Inc. of Edison, N.J., manufactures and sells alkoxylated esters of aromatic dicarboxylic and tricarboxylic acids, which are disclosed in U.S. Pat. Nos. 5,693,316, 5,597,555, 5,455,025, and 5,302,377. A broad class of useful emollients which are esters of aromatic alkoxylated alcohols and fatty carboxylic acids are also described and claimed in U.S. Pat. Nos. 6,987,195 and 7,217,424, both of which are also assigned to Croda, Inc.

Cyclomethicones are often used as ingredients in cosmetics and personal care products. They are generally clear, odorless, cyclic silicone based compounds known to impart a silky-smooth feel when applied to the skin. Cyclomethicones often stay well blended and are useful in wetting pigments, particles and fillers. Cyclomethicones also have generally low heat of vaporization. They also evaporate quickly after helping carry materials to the top layer of the epidermis.

There are a number of different structurally related cyclomethicones that are commonly used in cosmetics and personal care products. These include octamethylcyclotetracyloxane (D₄) [CAS No. 556-67-2], decamethylcyclopentacyloxane (D₅) [CAS No. 541-02-6], decamethylcyclohexacyloxane (D₆) [CAS No. 540-97-630]. While these widely used materials have long been considered safe, some are rethinking at least the prevalence of their use. Their overall stability and solubility profiles raise concerns that they could accumulate environmentally entering the food chain and thereby raise environmental and health issues. Indeed, some regulatory agencies such as, for example, the Canadian Government, are already considering restrictions on their use. David Gutierrez, “New Toxic Chemical in Cosmetics: D4 and D5 Siloxanes,” NaturalNews.com Apr. 6, 2009.

U.S. Pat. Nos. 6,987,195 and 7,214,424 disclose esters of alkoxylated aromatic alcohols and short chain fatty carboxylic acids and elsewhere exemplify cyclomethicone-free products with esters of alkoxylated aromatic alcohols and linear fatty carboxylic acids. See, inter alia, U.S. Pat. No. 7,217,424 col. 10 line 65 (PPG-6-phenoxyl caprate; col. 12 lines 5 (PPG-10 benzyl propionate) and 25 (PEG-10 benzyl acetate), and Examples 31-39, 41 and 42.

There is, however, still a need to find functional replacements for cyclomethicones and the present invention accomplishes just that.

### BRIEF SUMMARY OF TEE INVENTION

The present invention seeks to address these and other issues. It has been discovered that cyclomethicone-free cosmetics and personal care products, or those with a reduced content of a cyclomethicone, can be produced by using esters of aromatic alkoxylated alcohols and certain short chain carboxylic acids. Therefore, in one aspect of the present invention, there is provided a personal care or cosmetic product which is essentially true of cyclomethicone and in another embodiment, a product which is free (less than 1% by weight of the product). There is also provided a personal care or cosmetic product comprising: at least one ester of an aromatic alkoxylated alcohol and a carboxylic acid which is essentially cyclomethicone free or is cyclomethicone free (less than 1% by weight).

In another aspect there is provided a personal care or cosmetic product comprising: a) at least one ester of an aromatic alkoxylated alcohol and a short chain carboxylic acid. The ester is composed of (1) an alkoxylated aromatic alcohol comprising 1 to 20 alkoxy units selected from the group consisting of ethoxy (C₂), propoxy (C₃), and butoxy (C₄) units, the alkoxy group being bound, directly or through a connector, to the aromatic group with the resulting group including at least one free alcohol or hydroxy group, which can be used to condense with a short chain fatty carboxylic acid to form an ester, and (2) a short chain fatty carboxylic acid selected from the group consisting of a substituted or unsubstituted, saturated or mono-unsaturated, branched or cyclic fatty carboxylic acids of 4 to 20 carbons (including the carbonyl group); or a linear (straight chain), saturated or mono-unsaturated fatty carboxylic acids of 2 to 30 carbons including the carbonyl group; the ester being present in the amount of from about 0.1% to about 99% by weight of the product, and

b) at least one functional ingredient present in the amount of from about 0.1% to 60% by weight of the product. In one embodiment of this aspect of the invention, the cosmetic or personal care product consists essentially of these ingredients. In another embodiment, any of the forgoing cosmetic or personal care products are essentially free of a cyclomethicone. In still another embodiment, these products include less than about 1% of a cyclomethicone. In still another embodiment, these products include no more than about 10% of a cyclomethicone by weight but the ratio of the above-identified esters to the cyclomethicones is at least 2:1. Finally, in one embodiment of this aspect of the invention, the products exhibit similar feel properties (spreadability and wetness) compared to D₅ as measured by the rub out sensory assessment method using an expert panel.

In another aspect of the present invention, there is provided a personal care or cosmetic product comprising: a) at least one ester of an alkoxylated aromatic alcohol. The alkoxylated aromatic alcohol comprises 1 to 10 alkoxy units selected from the group consisting of ethoxy (C₂) and propoxy (C₃) units which are bound, directly or through a connector, to the aromatic or aromatic alcohol group, the resulting alkoxylated aromatic alcohol group including at least one free alcohol or hydroxy group. The ester also includes (2) a substituted or unsubstituted, saturated or mono-unsaturated, branched or cyclic fatty carboxylic acid of 5 to 12 carbons including the carbonyl group; the ester being present in the amount of from about 0.1% to about 99% by weight of the product, and

b) at least one functional ingredient present in the amount of from about 0.1% to 60% by weight of the product. In one embodiment of this aspect of the invention, the cosmetic or personal care product consists essentially of these ingredients. In another embodiment, any of the forgoing cosmetic or personal care products are essentially free of a cyclomethicone. In still another embodiment, these products include less than about 1% of a cyclomethicone. In still another embodiment, these products include no more than about 10% of a cyclomethicone by weight but the ratio of the above-identified esters to the cyclomethicones is at least 2:1. Finally, in one embodiment of this aspect of the invention, the products exhibit similar spreadability and wetness compared to Cyclomethione (D₅) as measured by the rub out sensory assessment method.

In still another aspect, any of these forgoing cosmetic or personal care products may be selected from the group consisting of sunscreens, sun blocks, antiperspirants, deodorants, and colored cosmetics.

In still another aspect of the present invention, there is provided a personal care or cosmetic product comprising: a) at least one ester of an alkoxylated aromatic alcohol and a carboxylic acid. The alkoxylated aromatic alcohol comprises an aromatic alcohol which is a substituted or unsubstituted benzyl, or a substituted or unsubstituted phenol bound, directly or through a connector, to an alkoxy group comprising 2 to 7 alkoxy units (monomers which form the group) selected from the group consisting of ethoxy (C₂) and propoxy (C₃) units and wherein at least one, and preferably a majority of alkoxy units are propoxy. In one particular embodiment of this aspect, the connector is itself an alkoxy unit. The resulting group will have at least one free alcohol or hydroxy group. The resulting alkoxylated aromatic alcohol is bound to a substituted or unsubstituted, saturated or mono-unsaturated, branched or cyclic fatty carboxylic acid of 5 to 8 carbons including the carbonyl group to form an ester; the ester being present in the amount of from about 0.1% to about 99% by weight of the product, and

b) at least one functional ingredient present in the amount of from about 0.1% to 60% by weight of the product. In one embodiment of this aspect of the invention, the cosmetic or personal care product consists essentially of these ingredients. In another embodiment, any of the forgoing cosmetic or personal care products are essentially free of a cyclomethicone. In still another embodiment, these products include less than about 1% of a cyclomethicone. In still another embodiment, these products include no more than about 10% of a cyclomethicone by weight but the ratio of the above-identified esters to the cyclomethicones is at least 2:1. Finally, in one embodiment of this aspect of the invention, the products exhibit similar spreadability and wetness compared to D₅ as measured by the rub out sensory assessment method.

In still another aspect, any of the forgoing cosmetic or personal care products are selected from the group consisting of sunscreens, sun blocks, antiperspirants, deodorants, and colored cosmetics.

It has been surprisingly discovered that while esters of aromatic alkoxylated alcohols and fatty carboxylic acids are known and are useful as emollients, some members of a relatively small subset of this broad class of compounds not only provides good emolliency, but can be used to reduce or eliminate the need for a cyclomethicone in certain finished products. Some of these materials can provide the silky feel, spreadability and desirable tactile application qualities such as low wetness so characteristic of cyclomethicones. In particular, some of these esters offer similar spreadability and wetness compared to D₅ as measured by the rub out sensory assessment method. However, they can be less volatile and will tend to stay on the skin. Finally, these materials have the particle wetting capabilities and solvent properties necessary to replace some or all of a cyclomethicone used in cosmetic or personal care products. This allows them be useful in the formulation of certain cosmetics and personal care products where a cyclomethicone would normally be used, including products that rely heavily on pigments, powdered and colored ingredients, fillers or other functional particles.

### DETAILED DESCRIPTION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. In the case of cyclomethicones, however, consisting essentially of generally means less than about 1% total cyclomethicones by weight of the product or formulation.

All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

“Essentially free of a cyclomethicone” or just “essentially free” as used herein means that a product or formulation includes at least one ester of an alkoxylated aromatic alcohol and a fatty carboxylic acid as described herein and less than 5% by weight of D₄, D₅, and D₆ or analog. “Free of a cyclomethicone” means that the amount of D₄, D₅, D₆, or an analog, is about 0.5% by weight or less.

The terms “personal care product or cosmetic product(s)” or more simply “product(s)” as used herein include, without limitation: aftershave lotions; baby lotions, oils, powders and creams; baby products, misc.; baby shampoos; basecoats and undercoats; bath capsules; bath oils, tablets and salts; bath preparations, misc.; bath soaps and detergents; beard softeners; blushers (all types); body and hand preparations (excluding shaving preparations); bubble baths; cleansing products (cold creams, cleansing lotions, liquids and pads); colognes and toilet waters; cuticle softeners; dentifrices (aerosol, liquid, pastes and powders); deodorants (underarm); depilatories; douches; eyebrow pencils; eyeliners; eye lotions; eye makeup preparations, misc.; eye makeup removers; eye shadows; face and neck preparations (excluding shaving preparations); face powders; feminine hygiene deodorants; foot powders and sprays; foundations; fragrance preparations, misc.; hair bleaches; hair coloring preparations, misc.; hair color sprays (aerosol); hair conditioners; hair dyes and colors (all types requiring caution statements and patch tests); hair lighteners with color; hair preparations (non-coloring), misc.; hair rinses (coloring); hair rinses (non-coloring), misc.; hair shampoos (coloring); hair sprays (aerosol fixatives); hair straighteners; hair tints; hair wave sets; indoor tanning preparations; leg and body paints; lipsticks; makeup bases; makeup fixatives; makeup preparations (not eye), misc.; manicuring preparations, misc.; mascara; men's talcum; moisturizing preparations; mouthwashes and breath fresheners (liquids and sprays); nail creams and lotions; nail extenders; nail polish and enamel removers; nail polish and enamels; night skin care preparations; oral hygiene products, misc.; paste masks (mud packs); perfumes; permanent waves; personal cleanliness products, misc.; powders (dusting and talcum, excluding aftershave talcs); preshave lotions (all types); rouges; sachets; shampoos (non-coloring); shaving cream (aerosol, brushless and lather); shaving preparations, misc.; shaving soap (cakes, sticks, etc.); skin care preparations, misc.; skin fresheners; suntan gels, creams, and liquids; suntan preparations, misc.; and tonics, dressings and other hair grooming aids.

In one particular embodiment, these terms mean sunscreens, sun blocks, terms which include other products which may contain sunscreens and/or sun blocks, deodorants, antiperspirants and colored cosmetics.

The term “colored cosmetic” as used herein means something meant to be left on the skin for more than just a few moments and impart color on or to the skin. These products often include pigments or other coloring materials. They include, without limitation: basecoats and undercoats, eyebrow pencils, eyeliners, eye lotions, eye makeup preparations, eye shadows, mascara, face and neck preparations (excluding shaving preparations), face powders, foundations, indoor tanning preparations, leg and body paints, lipsticks, lip liners, makeup bases, makeup fixatives, makeup preparations (not eye), nail polish and enamels, suntan gels, creams, and liquids, and suntan preparations.

The term “similar” in the context of skin feel tests means that panel tests for spreadability and wetness will result in assessments which are not less than about 20% of that obtained from identical panel assessments with cyclomethicon D₅. Assessments described herein were conducted by Sensory Spectrum, Inc., New Providence N.J.

The term “an ester of alkoxylated aromatic alcohol and fatty carboxylic acid,” which is used in defining the compounds of the invention, is the common name for a class of esters compounds described herein. It should be understood that the esters of the invention may be produced by any methods known in the art, rather than exclusively from the condensation of alkoxylated aromatic alcohols and fatty carboxylic acids.

An “alkyl” group is a group that includes a chain of carbon atoms. It includes alkanes, alkenes, and alkyns. “Lower alkyl” means an alkyl group comprising 6 or fewer carbons in total. “Short chain” means 30 carbons or less. Alkyl groups may be substituted or unsubstituted, straight chain or branched, unless specified otherwise. The term “fatty” in reference to alkyl groups and/or carboxylic acids is not intended to indicate origin. Rather, the term “fatty” is used to indicate the alkyl group of the carboxylic acids used in the esters of the invention. When a fatty carboxylic acid is identified herein by length or number of carbons, that number includes the carbonyl carbon unless otherwise stated.

Unless specified otherwise, fatty carboxylic acids may be branched, cyclic or straight chained, saturated or monounsaturated (one double bond). Cyclic means that some or all of the carbons in the fatty chain may form a non-aromatic ring which may be saturated or monounsaturated, substituted or unsubstituted as described herein. Without limitation, and for example only, these cyclic groups include C₆H₁₁—COO—, C₄H₆Cl—COO— and C₆H₁₁—CH₂COO— Substituted fatty acids may be substituted with one or more halogens (such as F, Cl, Br, I etc), hydroxyl, alkoxy, and oxyalkyl groups. Non limiting examples of branched fatty carboxylic acids useful in accordance with the invention include, without limitation, 2-ethyl hexanoic acid, neopentanoic acid, methyl pentanoic acids, 2-methyl hexanoic acid, valeric acid, caproic acid, and caprylic acid. In some embodiments, fatty acids useful in accordance with the present invention are linear and include 2-30 carbons including the carbonyl group. In other embodiments, the fatty acids are branched or cyclic and include 4-20 carbons including the carbonyl group. In still another embodiment, the fatty acids are branched or cyclic and include 5-12 carbons including the carbonyl group. In yet another embodiment, the fatty acids are branched or cyclic and include 5-8 carbons including the carbonyl group.

The “alkoxylated aromatic alcohols” used in the esters of the invention include those discussed herein and any disclosed in U.S. Pat. Nos. 6,987,195 and 7,217,424, the texts of which, and in particular, col. 4 line 39 through col. 7 line 15 of the '424 patent, are hereby incorporated by reference. Note that as used herein, the term alkoxylated aromatic alcohols can mean, as appropriate, a group that is an aromatic alcohol such as a benzyl alcohol, bound directly to the alkoxy group, a group that includes an aromatic alcohol bound to a connector which bridges between the aromatic alcohol and the alkoxy group, such as phenoxy ethanol, or it may result from the combination some other aromatic group, such as a benzyl group, with the connector. However, in all cases, the alkoxylated aromatic alcohol must include at least one free alcohol or hydroxy group which can be condensed to form an ester with the fatty carboxylic acid group.

In one embodiment, the aromatic nucleus of the alkoxylated aromatic alcohol may contain from 6 to 20 carbon atoms exclusive of substitution. Nuclei having 6 and 10 carbon atoms are preferred. Non-limiting examples of the aromatic nuclei are benzene, naphthalene and anthracene nuclei, which contain 6, 10 and 14 carbon atoms, respectively. In another embodiment, the aromatic nucleus is benzyl such as, for example only, C₆H₅—R, where R is a lower alkyl or H, or a benzyl alkyl alcohol such as, for example only, C₆H₅—CH₂—OH, C₆H₅—CH₂—CH₂—OH. In another embodiment, the aromatic nucleus is a phenol C₆H₅—OH.

These may be substituted or unsubstituted. Non-limiting examples of the substituents that may be bound to the aromatic nucleus, include lower alkyl (up to 6 carbons), such as methyl, ethyl, n-propyl, i-propyl, and butyl; aryl; arylalkyl; alkylaryl; halo, such as fluoro, chloro and bromo; acetoxy; alkylacetoxy; arylacetoxy; carboxy; alkylcarboxy; hydroxy; alkoxy; such as methoxy, ethoxy and propoxy; and alkylhydroxy.

The aromatic nucleus is connected to an alkoxy spacer, also referred to herein as an alkoxy group. The aromatic nucleus may be directly attached to the alkoxy group. For example, a benzyl alcohol can form an ester with the alkoxy group. However, in one embodiment, the nucleus is bound to a group which connects the nucleus to the alkoxy spacer. The connector group may be a straight-chain or branched, saturated or unsaturated chain of up to 6 carbons and may include one or more terminal oxygen or hydroxy groups (for non-limiting examples —CH₂CH₂CH₂—O—, —O—CH₂CH₂CH₂CH₂CH₂—, —CH═CH— and —CH₂CH₂). In particular, the connector may be an alkoxy group. For example, a phenoxy ethanol may be reacted with a chain including seven propoxy units. The resulting structure could be thought of as a phenol plus an alkoxy group of eight propoxy units or a phenol, a propoxy connector, and an alkoxy group of seven propoxy units. Thus, when phenol is used, the number of alkoxy units recited may be extended by one, or one of the alkoxy units can be considered a connector.

Non-limiting examples of the nuclei are C₆H₅—O—, C₆H₅—C_{H}2-O—,

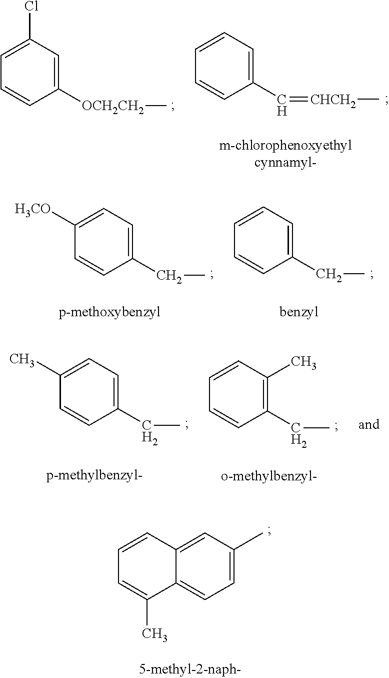

Non-limiting examples of aromatic nuclei are

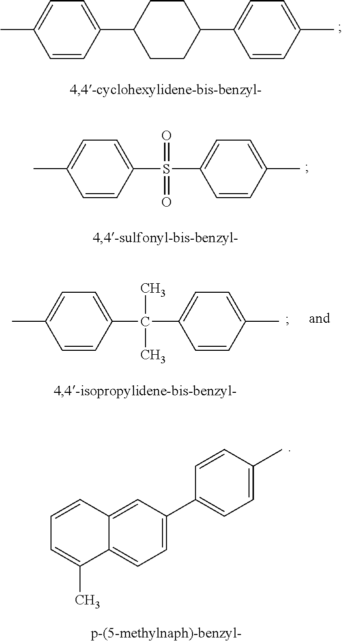

Another non-limiting example of an aromatic nuclei is

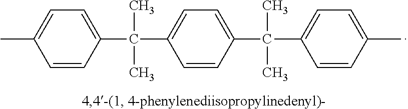

The alkoxy group or spacer is made up of from 1 to 20 alkoxy units which may be same or different. Preferably, the alkoxy spacer includes x number of ethoxy units and y number of propoxy units, which may be present in any order, such as in blocks, randomly or in alternating pattern where the total of x and y is 1 to 20. In describing the esters of the invention, the structure of the alkoxy spacer may be shown as, for example,

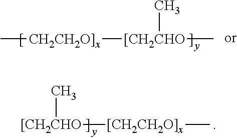

Such depiction indicates only the number of ethoxy and propoxy units, and does not indicate their order or the nature of attachment of the alkoxy spacer to the group nucleus or the ester group unless indicated otherwise. For example, the spacer shown as

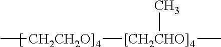

does not indicate a block of 4 ethoxy groups attached to a block of 4 propoxy groups. It does mean, however, that an alkoxy spacer having 4 ethoxy and 4 propoxy units is contemplated.

In another embodiment, the sum of x and y ranges from 1 to 10, inclusive and in another embodiment from 1 to 5. In one instance, x is equal to 0 and in another y is equal to 0. X could be 1 or 2 while y is equal to 0 or y could be 1 or 2 while x is equal to 0. In yet another group of such esters, x>0, y>0, and x>y. In yet another group of such esters, x>0, y>0, and y>x.

In some embodiments, the number of alkoxy units which make up the alkoxy group ranges from 1 to 20 which can be ethoxy units, propoxy units, or butoxy units. In some embodiments, there is at least one propoxy unit in the alkoxy group. In another embodiment, the alkoxy group includes a majority of propoxy groups. In one embodiment, all of the alkoxy units in the alkoxy group is propoxy. Where the groups include propoxy or butoxy units, those units may be linear or branched. In one embodiment, they include at least one branched unit. In another, the alkoxy group includes a majority of branched units. In another, the alkoxy group include all branched units.

In other embodiments, the number of alkoxy units which make up the alkoxy group ranges from 1 to 10 which can be ethoxy units or propoxy units. In some embodiments, there is at least one propoxy unit in the alkoxy group. In another embodiment, the alkoxy group includes a majority of propoxy groups. In one embodiment, all of the alkoxy units in the alkoxy group is propoxy. Where the groups include propoxy units, those units may be linear or branched. In one embodiment, they include at least one branched unit. In another, the alkoxy group includes a majority of branched units. In another, the alkoxy group include all branched units.

In still another embodiments, the number of alkoxy units which make up the alkoxy group ranges from 2 to 7 which can be ethoxy units or propoxy units. In some embodiments, there is at least one propoxy unit in the alkoxy group. In another embodiment, the alkoxy group includes a majority of propoxy groups. In one embodiment, all of the alkoxy units in the alkoxy group is propoxy. Where the groups include propoxy units, those units may be linear or branched. In one embodiment, they include at least one branched unit. In another, the alkoxy group includes a majority of branched units. In another, the alkoxy group include all branched units.

Alkoxylated aromatic alcohols useful in accordance with the present invention include, without limitation, alkoxylated benzyl alcohol, alkoxylated phenoxyethanol, alkoxylated cynnamyl alcohol, alkoxylated phenoxy-n-propanol, alkoxylated phenoxy-i-propanol, alkoxylated bisphenol A, alkoxylated bisphenol AF, alkoxylated bisphenol AP, alkoxylated tetramethyl bisphenol A, alkoxylated bisphenol F, alkoxylated bisphenol E, alkoxylated bisphenol C, alkoxylated bisphenol M, alkoxylated bisphenol P, alkoxylated bisphenol S, alkoxylated bisphenol Z and mixtures thereof.

The esters of the invention may be prepared in a variety of ways such as reacting:

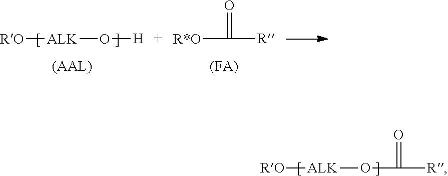

wherein AAL is an alkoxylated aromatic alcohol as described herein, and FA is a short chain, fatty carboxylic acid as defined herein. The various “R” groups illustrated are meant to represent the various hydrogen, alkyl or fatty groups spelled out herein for the esters, alkoxy groups and acids described herein as is known generally to chemists. They may all be the same or different as described herein.

The synthesis of an alkoxylated aromatic alcohol (AAL) typically involves alkoxylation of the corresponding aromatic alcohol. The reaction schemes below illustrate the alkoxylation reaction, showing the ethoxylation/propoxylation of benzyl alcohol:

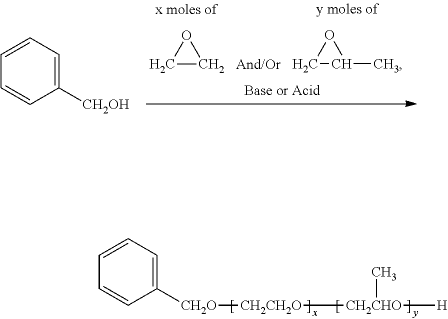

and p-di-methylhydroxy benzyl diol:

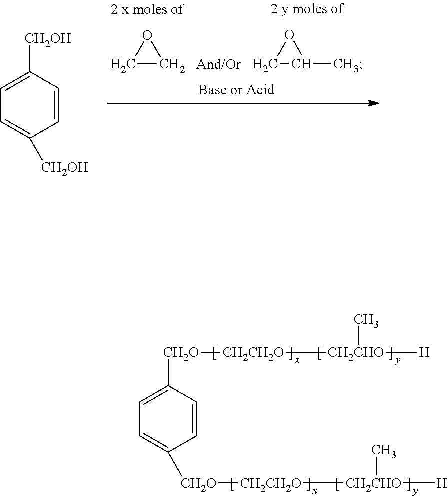

Preferably, the alkoxylation is carried out with ethylene oxide, propylene oxide or mixtures of ethylene oxide and propylene oxide. The alkoxylation of mono-hydroxy aromatic alcohols (e.g., benzyl alcohol, cynnamyl alcohol, phenoxyethanol, etc.) provides alkoxylated aromatic alcohols having a single hydroxy group at the end of the alkoxy chain. The alkoxylation of di-hydroxy aromatic alcohols typically provides alkoxylated aromatic alcohols having two hydroxy groups at the end of each alkoxy chain.

The alkoxylation reaction may produce symmetrical or asymmetrical alkoxylated alcohols. The order of the addition of the alkyl oxides (e.g., ethyl oxide and propyl oxide) may be used to vary the nature of the alkoxy substitution in the final esters, including the structural order of ethoxy and propoxy units, and the order of attachment. For example, alkylene oxides may be added sequentially, in a mixture in blocks, or in any other desired manner, thus varying the order of the alkoxy substitution. The amounts of alkyl oxide(s) introduced in the reaction zone and the duration of the reaction with the selected alkyl oxide(s) determines the number of moles of the alkyl oxide added to the starting aromatic alcohol. The numbers of ethoxy and propoxy units in the ester effects its properties. Thus, an increase in the amount of ethoxylation may be used to improve the solubility/dispersibility of the ester while an increase in the amount of propoxylation improves fluidity.

The alkoxylation reaction may proceed via acidic or basic catalysis in the presence of such catalysts as alkali hydroxides and oxides, protic and Lewis acids, amines, quaternary ammonium compounds, water, and catalysts mixtures. The specific suitable catalysts include, for example, potassium hydroxide, sodium methoxide, sodium borohydride, boron trifluoride, stannic chloride, and sulfuric acid. The preferred catalysts are potassium or sodium hydroxide, sodium methoxide, sodium borohydride or mixtures thereof.

Preferably, the catalyst is used in the amount of from about 0.1 to about 2.0 weight percent of the weight of the desired alkoxylated alcohol. Preferably, the reaction is carried out under anhydrous conditions at from about 110° C. to about 200° C. at pressures of from about 10 psig to about 80 psig. Higher temperatures and pressures may be also utilized. After the reaction is complete, an acid (e.g., phosphoric or acetic acid) may be introduced to neutralize the catalyst.

The alkoxylated aromatic alcohols produced in the alkoxylation reaction can be esterified, providing the esters of alkoxylated aromatic alcohols and short chain fatty carboxylic acid of the invention. The esterification reaction may be carried out in a conventional manner. Typically, the stoichiometric quantities of the alcohol and the fatty acid are combined in the presence of a suitable catalyst. A slight stoichiometric excess of one of the reagents may be employed. The preferred catalysts include methanesulfonic acid and paratoluenesulfonic acid.

Generally, the aromatic alkoxylated alcohol, the fatty carboxylic acid and the catalyst are combined with mixing to form a reaction mixture. The reaction mixture is heated with mixing at a temperature between about 155° C. and about 250° C., preferably between about 170° C. and 220° C., until an acid value of less than 8 mg KOH, preferably less than 5 mg KOH, is obtained. The reaction mixture is cooled below 85° C. and washed with water. The ester layer is separated and heated under vacuum until moisture content of less than 0.2 percent is obtained.

The esterification of alkoxylated aromatic alcohols having a single hydroxy group is straightforward. If a stoichiometric excess of the fatty acid or the derivative of fatty acid is provided, the alkoxylated aromatic alcohol is entirely consumed in the reaction, providing a monoester of alkoxylated aromatic alcohol and the fatty acid. The reaction scheme below illustrates the esterification of PEG-4, PPG-4 benzyl ether by myristic acid, providing 4-PEG-4-PPG benzyl myristate:

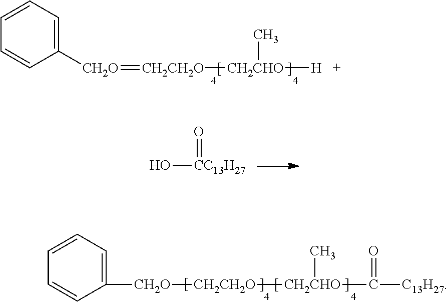

The esterification of the alkoxylated aromatic alcohols having two or more hydroxy groups capable of being esterified may result in monoesters, diesters and so on. The nature of the esterification product depends on the relative proportions of the fatty carboxylic acid (or the derivative thereof) and the alkoxylated aromatic alcohol. For example, the esterification of alkoxylated aromatic alcohols having two hydroxy groups may result in one or both of the hydroxy groups being esterified, as desired. If a stoichiometric excess of the alkoxylated aromatic alcohol is present in the reaction mixture, the monoester is the predominant product. Conversely, if a stoichiometric excess of the fatty acid is used, the predominant product is the diester.

The reaction scheme below illustrates the esterification of PEG-4, PPG-4 p-di-methylhydroxy benzyl ether by myristic acid, providing either di- or mono-myristate depending on the relative proportions of the reactants:

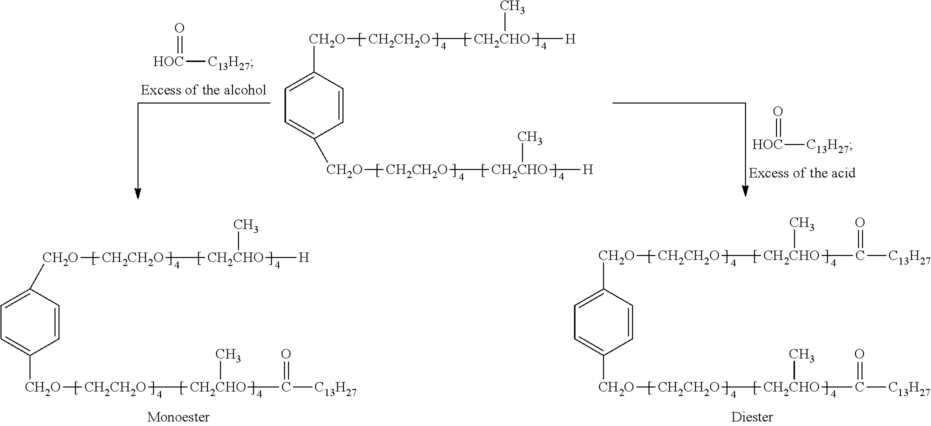

The descriptions of the preparation methodologies, including the alkoxylation and esterification reactions, in U.S. Pat. Nos. 5,693,316, 5,597,555, 5,455,025, and 5,302,377 are incorporated herein by reference to the extent such descriptions are relevant to the preparation of the esters of the invention.

The esters of alkoxylated aromatic alcohols and short chain fatty carboxylic acids are useful as ingredients or additives for cosmetic and personal care products generally, but in particular generally cyclomethicone-free cosmetics or personal care products. Preferably, the esters described herein are utilized as emollients, although they may be used as solubilizers, diluents, plastisizers, and/or thickeners. As ingredients of personal care and cosmetic products, the esters of alkoxylated aromatic alcohols and fatty carboxylic acids may provide one or more important advantages to the formulator. First of all, the formulations containing the esters of the invention can have a non-greasy skin feel desirable for cosmetic and personal care products. This, of course, depends on other ingredients of the formulation. Second, some formulations containing these esters would be more environmentally friendly as at least some of these esters are biodegradable. Thirdly, many of these esters can impart shine or gloss, and when added to other gloss agents such as silicone, they can change the refractive indices of these materials. Finally, many of these materials have wetting properties that aid in the dispersing of solid ingredients such as many of the materials used in color cosmetics, sunscreens and antiperspirant/deodorant products.

It is another advantage that the synthetic methodology for preparation of the esters of the invention provides ability to modify the properties of the cosmetic and personal care product containing the esters. For example, by modifying the ratios of ethoxy and propoxy groups in the alkoxy spacer, the fluidity and/or dispersibility of the esters in surfactant systems may be varied as the formulator desires. To impart additional fluidity, the propoxy units are included in the alkoxy spacer of the ester. Conversely, the ethoxy units may be included to improve the solubility/dispersibility of the esters.

Compounds that impart the products with low spreading properties allow formation of thicker, more uniform films. Such compounds are desirable for obtaining, for example, sunscreen formulations with a high resistance to wash off and a high SPF value. Therefore, the esters of alkoxylated aromatic alcohols and fatty carboxylic acids having a skin spread factor of less than 10 or a viscosity of less then 2,000 cps are preferred. The more preferred esters have a skin spread factor of less than 8 or a viscosity of less then 1,000 cps. The esters that exhibit both low spread factor and viscosity are yet more preferred.

The skin spread factor is determined by applying, for example, 5 microliters of a product to a 3.14 cm diameter circle on the volar surface of the forearm. The product is spread evenly within the circle and allowed to spread for a fifteen minute period. The test site and surrounding area of the forearm is then sprayed with a 1 percent solution of FD&C Blue No. 1. The area not stained by the blue dye indicates the area onto which the product spread. The skin-spread factor is obtained by dividing the resulting area by the initial area. In accordance with the present invention, the test formulations containing the esters of the invention preferably have a skin spread factor of less than about 10, more preferably less than about 5.

Desirable pigment wetting properties may also be obtained by using the esters of the invention. Pigment wetting properties are determined by preparing a dye slurry and measuring viscosity. For example, a slurry can be prepared using an alkoxylated aromatic alcohol fatty esters of the present invention mixed with 35 percent mica. The viscosity of the slurry is determined after allowing the slurry to stand for five minutes. The lower the viscosity, the better the pigment wetting properties of the ester. Preferably, in accordance with the present invention, viscosity of the resulting dye slurry will be below about 20,000 cps, more preferably, less than about 10,000 cps; most preferred less than 2,000; based on a 65% ester, 35% pigment mixture (measured in weight percent). Viscosity will vary if a different testing mixture is used.

Thus, the invention may provide personal care or cosmetic products that include the esters of alkoxylated aromatic alcohols and fatty carboxylic acids described herein. The final products of the invention may contain from about 0.1% to about 99% of at least one ester of alkoxylated aromatic alcohol and short chain fatty carboxylic acid by weight of the composition. The amount of the ester(s) in the products depend on the specific application and formulation design. Preferably, the esters of the invention are present in the amount from about 0.1% to about 40%, more preferably, from about 0.1% to about 10%, yet more preferably, from about 0.5% to about 2% by the weight of the products and composition. However, different amounts may be preferred depending on the nature of the product.

In addition to the esters of alkoxylated aromatic alcohols and short chain fatty carboxylic acids of the invention, the final products of the invention may include various functional ingredients, including active and additional ingredients, both conventional and otherwise. The decision to include or exclude an ingredient and the selection of specific functional ingredients depends on the specific application and product formulation. The line of demarcation between an “active” ingredient and an “additional ingredient” is artificial and dependent on the specific application and product type. A substance that is an “active” ingredient in one application or product may be an “additional” ingredient in another, and vice versa.

Thus, in addition to the esters of alkoxylated aromatic alcohols and short chain fatty carboxylic acids of the invention, the products of the invention may include one or more functional ingredients present in the amount from 0.1% to about 60%, preferably, from about 3% to about 40%, more preferably, from about 5% to 25% by weight of the composition. The active ingredients provide some benefit related to the function of the product. Non-limiting examples of the functional ingredients include pharmaceutical ingredients, antidandruff ingredients, abrasives, absorbents, adhesives, antiacne agents, anticaking agents, anticaries agents, antidandruff agents, antifoaming agents, antifungal agents, antimicrobial agents, antioxidants, antiperspirant agents, antistatic agents, binders, buffering agents, bulking agents, chelating agents, colorants, corn/callus/wart removers, corrosion inhibitors, cosmetic astringents, cosmetic biocides, denaturants, deodorant agents, depilating agents, drug astringents, drug astringents—oral health care drugs, drug astringents—skin protectant drugs, emulsion stabilizers, epilating agents, exfoliants, external analgesics, film formers, flavoring agents, fragrance ingredients, hair colorants, hair conditioning agents, hair fixatives, hair-waving/straightening agents, humectants, lytic agents, nail conditioning agents, opacifying agents, oral care agents, oral healthy care drugs, oxidizing agents, pesticides, pH adjusters, plasticizers, preservatives, propellants, reducing agents, skin bleaching agents, skin-conditioning agents, skin-conditioning agents—emollient, skin-conditioning agents—humectant, skin-conditioning agents—miscellaneous, skin-conditioning agents—occlusive, skin protectants, slip modifiers, solvents, sunscreen agents, surface modifiers, surfactants, surfactants—cleansing agents, surfactants—emulsifying agents, surfactants—foam boosters, surfactants—hydrotropes, surfactants—solubilizing agents, surfactants—suspending agents, suspending agents—nonsurfactant, ultraviolet light absorbers, viscosity controlling agents, viscosity decreasing agents, viscosity increasing agents—aqueous, and viscosity increasing agents—nonaqueous. Other suitable functional ingredients and the specific types of the functional ingredients can be found in the general ingredients section herein.

For each type of functional ingredient, one or more compounds may be present. Likewise, more than one type of functional ingredient may be present in the product composition. The choice of the functional ingredient depends on the nature of the desired cosmetic or personal care product. For example, the sunscreens may be used in the sunscreen lotions, shampoos, hair care lotions and the like. The choice of the functional ingredient(s) depends on the nature of the desired cosmetic or personal care product. For example, the sunscreen actives may be used in the sunscreen lotions, shampoos, medicated shampoos, hair care lotions and the like.

In addition to esters of alkoxylated aromatic alcohols and short chain fatty carboxylic acids and the active ingredient(s), the compositions of the invention may include one or more additional emollients, detergents, emulsifiers, humectants, antioxidants, softeners, lubricants, penetrants, plastisizers, solvents and co-solvents, sunscreening additives, dispersants, antiperspirants, conditioners, thickening agents, preservatives, antimicrobial agents, buffers, chelating agents, foaming agents, pigments, colorants, fillers and particulates, coupling agents, proteins, salts, essential oils and fragrances.

The final products of the invention may be in the form of liquids, gels, creams, emulsions, aerosol and non-aerosol sprays, microemulsions, foams, sticks and solids, and may be clear or opaque. Clear preparations are preferred. The final product compositions of the invention, including those in the form of products described herein, may be used for both skin and hair.

The final products of the invention may be formulated as aqueous and non-aqueous preparations, including but not limited to topical preparations.

The aqueous topical preparations of the invention contain the esters of alkoxylated aromatic alcohols and short chain fatty carboxylic acids the invention in the amount of from about 0.10 to about 60.0 percent, preferably from about 3.0 to about 40.0 percent, more preferably, 5 to 25 percent by weight of the composition, and one or more active ingredients in the amount from about 0.10 to about 60.0 percent, preferably from about 3.0 to about 40.0, more preferably, 5 to 30 percent by weight of the composition. If additional conventional emollients are used in the composition, they may be present in a ratio of from about 10:1 to about 1:10 parts to the ester compounds of the invention. The balance is generally excipients and water or some aqueous solvent system.

The non-aqueous topical preparations of the invention contain the esters of alkoxylated aromatic alcohols and short chain fatty carboxylic acids in the amount of from about 0.10 to about 99.0 percent, preferably from about 10 to about 90.0 percent, and more preferably from about 15 to about 75 percent by weight of the composition, and one or more of the active ingredients in the amount from about 0.20 to about 99.0 percent, preferably, from about 10 to about 90.0 percent, and more preferably, from about 15 to about 75 percent by weight of the composition. If additional conventional emollients are used in the composition, they may be included in a ratio of from about 10:1 to about 1:10 to the ester(s) of the invention.

Non-limiting examples of the cosmetic personal care products of the invention include products which would often include a cyclomethicone, such as sunscreen compositions for hair and/or skin, such as lotions, gels, sprays, and the like, suntan oils, antiperspirant and/or deodorant compositions, cold creams, deodorants, topical pharmaceutical ointments, skin moisturizers, cleansing creams, skin lotions and creams, hair conditioners, detergents, make-up products, lipstick products, mascara.

The antiperspirants product compositions containing the esters of alkoxylated aromatic alcohols and fatty carboxylic acids may also contain a variety of other ingredients, including those traditionally included. Non-limiting examples of such ingredients are aluminum zirconium complexes, such as aluminum zirconium tetrachlorohydrex-glycine; aluminum chlorohydrates; sodium aluminum chlorohydroxy lactate; aluminum chlorohydrex P.G.; glycols, such as dipropylene glycol, hexylene glycol and the like; silicones, such as phenyl trimethicone; talc and the like; hydroxyethyl cellulose and the like; PEG distearate and the like; fatty esters, such as isopropyl myristate and the like; alkoxylated alcohols, such as isoceteth-20 and the like; ethanol; polydecene and the like; polyethylene; silica; fatty alcohols, such as cetyl, stearyl and cetearyl; hydrogenated vegetable oils, such as hydrogenated castor oil and the like; glyceryl behenate; C₁₈₋₃₆ acid triglycerides; and C₁₈₋₃₆ acid glycol esters. Other suitable specific ingredients are described herein in the general ingredients section.

The esters of the invention are also especially useful as ingredients of sunscreen product formulations, such as sunscreen lotions, sunscreen lotion sprays, UV-protecting shampoos and conditioners, cosmetic formulations having a sun-protection function, and the like. Thus, the invention also provides sunscreen product compositions that include the esters of the invention present in the amount of from about 0.5% to about 60%, more preferably, from about 1% to about 40% by weight of the composition, and one or more sunscreen active ingredients present in the amount of from about 0.1% to 30%, more preferably, from about 1% to about 20% by weight of the composition. Non-limiting examples of the sunscreen active ingredients that may be used in the sunscreen product formulations of the invention include p-amino benzoates, salicylates, ferrulic acid derivatives, phenylbenzimidazole sulfonic acids, benzophenone sulfonic acids, benzophenones, thioctic acids derivatives, oil-soluble cinnamates, and the like. The sunscreen active ingredients present in the sunscreen products of the invention include nonionic, cationic and anionic UV-absorbing compounds, such as benzophenone-3, octyl methoxycinnamate, phenylbenzimidazole sulfonic acid, menthyl anthranilate, cinnamidopropyl trimonium chloride and the like. Other specific non-limiting examples of the sunscreen active ingredients include para-aminobenzoic acid, benzophenone-1, benzophenone-1, benzophenone-2, benzophenone-4, benzophenone-5, benzophenone-6, benzophenone-8, benzophenone-12, methoxycinnamate, avobenzone, ethyl dihydroxypropyl para-aminobenzoate, glyceryl para-aminobenzoate, methyl anthranilate, octocrylene, octyl dimethyl para-aminobenzoate, octyl salicylate, zinc oxide, titanium dioxide, and red petrolatum. In one preferred embodiment, the esters of the formula (II) are used in combination with avobenzone.

The sunscreen product compositions containing the esters of alkoxylated aromatic alcohols and fatty carboxylic acids may also contain a variety of other ingredients, including those traditionally included. Non-limiting examples of such ingredients, which may be present in the sunscreen product compositions of the invention, are mineral oil; fatty acid esters, such as octyl palmitate and the like; emulsifiers, such as oleth-10, PEG-10 cetyl alcohol and the like; non-alkoxylated alcohol phosphate esters, such as cetyl phosphate or dicetyl phosphate; alkoxylated alcohol phosphate esters, such as PEG-10 cetyl phosphate and the like; polymeric emulsifiers, such as carbomer-type and the like; physical blockers, such as zinc oxide and titanium dioxide; fatty alcohols, such as cetyl, stearyl and cetearyl; vitamins, such as vitamin E, C and the like; antioxidants, such as BHT, BHA, and the like. Other suitable specific ingredients are described herein in the general ingredients section.

The following ingredients also may be present in the compositions of the invention.

Surfactants

Various surfactants may be present in the compositions of the invention, including one or more nonionic surfactants, anionic surfactants, cationic surfactants, amphoteric surfactants, zwitterionic surfactants, and mixtures thereof. For some of surfactants that may be used in combination with the compositions of the invention, please see McCutcheon's, Detergents and Emulsifiers, (1986), U.S. Pat. Nos. 5,151,210, 5,151,209, 5,120,532, 5,011,681, 4,788,006, 4,741,855, U.S. Pat. Nos. 4,704,272, 4,557,853, 4,421,769, 3,755,560; all incorporated herein by reference in their entirety.

Cationic Surfactants

Various cationic surfactants may be present in the compositions of the invention. The amounts and the nature of cationic surfactants present in the compositions of the invention depend on the nature of the composition. In the final product composition, the total amount of cationic surfactants, including the alkoxylated esters thereof described herein, may vary from 0.1% to about 40%, more preferably, from about 0.1% to about 15%, yet more preferably, from about 0.5% to about 2% by the weight of the product composition. However, different amounts of cationic surfactants may be preferred depending on the nature of the product. Suitable additional cationic surfactants are disclosed in McCutcheon, Detergents & Emulsifiers, (M.C. Publishing Co. 1979); U.S. Pat. Nos. 3,155,591, 3,929,678, 3,959,461, 4,387,090, which are incorporated by reference herein.

Ammonium Quats

The compositions of the invention may include quaternary ammonium cationic surfactants of the formula

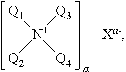

where X and a are as previously described, Q₁ is C₁₂-C₂₂ alkyl, C₁₂-C₂₂ alkyl amido C₁-C₆ alkylene, C₁₂-C₂₂ alkylhydroxy; Q₂ is C₁₂-C₂₂ alkyl, C₁₂-C₂₂ alkyl amido C₁-C₆ alkylene, C₁₂-C₂₂ alkylhydroxy, benzyl, or C₁-C₆ alkyl; Q₃ and Q₄ are independently C₁-C₆ alkyl or benzyl.

Examples of suitable quaternary ammonium surfactants include cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl dimethyl ammonium chloride, distearyl methyl ammonium bromide, and mixtures thereof.

Additional quaternary ammonium salts include those wherein the C₁₂-C₂₂ alkyl is derived from a tallow fatty acid or from a coconut fatty acid. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconut alkyl)dimethyl ammonium chloride, di(coconut alkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

More preferred quaternary ammonium surfactants are dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Fatty Amines

The compositions of the invention may also include salts of primary, secondary and tertiary C₁₂-C₂₂ amines. Examples of such suitable amines include stearamido propyl dimethyl amine, diethyl amino ethyl stearamide, dimethyl stearamine, dimethyl soyamine, soyamine, tri(decyl)amine, ethyl stearylamine, ethoxylated stearylamine, dihydroxyethyl stearylamine, and arachidylbehenylamine. Suitable amine salts include the halogen, acetate, phosphate, nitrate, citrate, lactate and alkyl sulfate salts. Such salts include stearylamine hydrochloride, soyamine chloride, stearylamine formate, N-tallowpropane diamine dichloride and stearamidopropyl dimethylamine citrate. Some cationic amine surfactants useful in the compositions of the present invention are disclosed in U.S. Pat. No. 4,275,055, incorporated by reference herein.

Amidoamines

The compositions of the invention may also include amidoamines, such as disclosed in U.S. patent application Ser. No. 09/409,203, assigned to Croda Inc., and incorporated by reference herein.

Non-Ionic Surfactants

The compositions of the invention may also include various non-ionic surfactants. Among the suitable nonionic surfactants are condensation products of C₈-C₃₀ alcohols with sugar or starch polymers. These compounds can be represented by the formula (S)ₙ—O—R, wherein S is a sugar moiety such as glucose, fructose, mannose, and galactose; n is an integer of from about 1 to about 1000, and R is C₈-C₃₀ alkyl. Examples of suitable C₈-C₃₀ alcohols from which the R group may be derived include decyl alcohol, cetyl alcohol, stearyl alcohol, lauryl alcohol, myristyl alcohol, oleyl alcohol, and the like. Specific examples of these surfactants include decyl polyglucoside and lauryl polyglucoside.

Other suitable nonionic surfactants include the condensation products of alkylene oxides with fatty acids (i.e., alkylene oxide esters of fatty acids). These materials have the general formula RCO(X)ₙ OH, wherein R is a C₁₀-C₃₀ alkyl, X is —OCH₂CH₂— (derived from ethylene oxide) or —OCH₂CHCH₃— (derived from propylene oxide), and n is an integer from about 1 to about 200.

Yet other suitable nonionic surfactants are the condensation products of alkylene oxides with fatty acids (i.e., alkylene oxide diesters of fatty acids) having the formula RCO(X)ₙOOCR, wherein R is a C₁₀-C₃₀ alkyl, X is —OCH₂CH₂—(derived from ethylene oxide) or —OCH₂CHCH₃— (derived from propylene oxide), and n is an integer from about 1 to about 200.

Yet other nonionic surfactants are the condensation products of alkylene oxides with fatty alcohols (i.e., alkylene oxide ethers of fatty alcohols) having the general formula R(X)ₙOR′, wherein R is C₁₀-C₃₀ alkyl, n is an integer from about 1 to about 200, and R′ is H or a C₁₀-C₃₀ alkyl.

Still other nonionic surfactants are the compounds having the formula RCO(X)ₙOR′ wherein R and R′ are C₁₀-C₃₀ alkyl, X is —OCH₂CH₂— (derived from ethylene oxide) or —OCH₂CHCH₃— (derived from propylene oxide), and n is an integer from about 1 to about 200.

Examples of alkylene oxide-derived nonionic surfactants include ceteth-1, ceteth-2, ceteth-6, ceteth-10, ceteth-12, ceteraeth-2, ceteareth6, ceteareth-10, ceteareth-12, steareth-1, steareth-2, stearteth-6, steareth-10, steareth-12, PEG-2 stearate, PEG4 stearate, PEG6 stearate, PEG-10 stearate, PEG-12 stearate, PEG-20 glyceryl stearate, PEG-80 glyceryl tallowate, PPG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Still other useful nonionic surfactants include polyhydroxy fatty acid amides disclosed, for example, in U.S. Pat. Nos. 2,965,576, 2,703,798, and 1,985,424, which are incorporated herein by reference.

Anionic Surfactants

The compositions of the invention may also include various anionic surfactants. Several examples of suitable anionic surfactants are disclosed in U.S. Pat. No. 3,929,678, which is incorporated herein by reference. Further examples of suitable anionic surfactants include alkoyl isethionates, and alkyl ether sulfates.

The alkoyl isethionates typically have the formula RCO—OCH₂CH₂—SO₃M, wherein R is C₁₀-C₃₀ alkyl, and M is a water-soluble cation, such as ammonium, sodium, potassium, or triethanolamine. The examples of suitable isethionates include ammonium cocoyl isethionate, sodium cocoyl isethionate, sodium lauroyl isethionate, sodium stearoyl isethionate, and mixtures thereof. Preferred for used herein are ammonium cocoyl isethionate, sodium cocoyl isethionate, and mixtures thereof.

The alkyl ether sulfates typically have the formulas ROSO₃M and RO(C₂H₄O)ₓSO₃M, where R is C₁₀-C₃₀ alkyl, x varies from about 1 to about 10, and M is a water-soluble cation such as ammonium, sodium, potassium and triethanolamine.

Yet another suitable class of anionic surfactants are alkali metal salts of C₈-C₃₀ carboxylic acids and alkylsulfonates of the formula R₁—SO₃M (where R₁ is C₈-C₃₀ alkyl; preferably, C₁₂-C₂₂ alkyl, and M is a cation), including succinamates, and C₁₂-C₂₄ olefin sulfonates and carboxylates.

Amphoteric Surfactants

The compositions of the invention may also include zwitterionic and amphoteric surfactants. Suitable amphoteric and zwitterionic surfactants are, for example, derivatives of mono- or di-C₈-C₂₄ secondary and tertiary amines, such as alkyl imino acetates, carboxylates, sulfonates, sulfates, phosphates, and phosphonates, including iminodialkanoates and aminoalkanoates of the formulas RN(CH₂)ₘ CO₂ M₂ and RNH(CH₂)ₘ CO₂M, where m varies from 1 to 4, R is C₈-C₃₀ alkyl; preferably, C₁₂-C₂₂ alkyl, and M is H, alkali metal, alkaline earth metal ammonium, or alkanolammonium.

Other suitable amphoteric and zwitterionic surfactants are imidazolinium and ammonium derivates. Suitable examples of such amphoteric surfactants include sodium 3-dodecyl-aminopropionate, sodium 3-dodecylaminopropane sulfonate, N-alkyltaurines; N-higher alkyl aspartic acids, and coamidopropyl PG-dimonium chloride phosphate. For further examples of suitable amphoteric and zwitterionic surfactants, please see U.S. Pat. Nos. 2,658,072, 2,438,091, and 2,528,378, which are incorporated herein by reference

Yet other suitable amphoteric and zwitterionic surfactants are betaines. Examples of suitable betaines include coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and amidobetaines and amidosulfobetaines, oleyl betaine, and cocamidopropyl betaine.

Sunscreen Compounds

A wide variety of sunscreen compounds are suitable for use with the compositions of the present invention. Depending on the nature of the composition, the sunscreen compounds may be added in the amount of up to about 40% by weight of the composition, preferably, from about 1% to about 30%. However, the preferred amount may vary depending on the nature of the composition. Thus, for the final product compositions in the form of a shampoo or conditioner, the suitable sunscreen agent may be included in the amount of up to about 40% by weight of the composition, preferably, from about 0.5% to about 5%, more preferably, from about 05 to about 1.5% by weight of the composition. Suitable sunscreen compounds include, for example, p-aminobenzoic acid, its salts and its derivatives; anthranilates; salicylates; cinnamic acid derivatives; dihydroxycinnamic acid derivatives; trihydroxycinnamic acid derivatives; hydrocarbons; dibenzalacetone and benzalacetophenone; naphtholsulfonates; dihydroxy-naphtholic acid and its salts; coumarin derivatives; diazoles; quinine salts; quinoline derivatives; hydroxy- or methoxy-substituted benzophenones; uric and vilouric acids; tannic acid and its derivatives; hydroquinone; amino benzoates, salicylates, ferrulic acid derivatives, phenylbenzimidazole sulfonic acids, benzophenone sulfonic acids, thioctic acids derivatives, oil-soluble cinnamates, and benzophenones. For other suitable sunscreen compounds, please see Segarin, et al., Cosmetics Science and Technology, Chapter VIII, pages 189 et seq., incorporated herein by reference.

Specific suitable sunscreen compounds include 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl-4->bis(hydroxypropyl)!-aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl p-dimethylaminobenzoate, 2-phenylbenzimidazole-5-sulfonic acid, 2-(p-dimethyl-aminophenyl)-5-sulfonicbenzoxazoic acid, para-aminobenzoic acid, benzophenone-1, benzophenone-1, benzophenone-2, benzophenone-3, benzophenone-4, benzophenone-5, benzophenone-6, benzophenone-8, benzophenone-12, methoxycinnamate, avobenzone, ethyl dihydroxypropyl para-aminobenzoate, glyceryl para-aminobenzoate, methyl anthranilate, octocrylene, octyl dimethyl para-aminobenzoate, octyl methoxycinnamate, octyl salicylate, zinc oxide, titanium dioxide, and red petrolatum.

Emollients

The compositions of the invention may also include one or additional emollient compounds such as fats, waxes, lipids, silicones, hydrocarbons, fatty alcohols and a wide variety of solvent materials. The amount of the emollient depends on the application. For the final product compositions, emollients are generally included in the amount of up to 50% by weight of the composition, preferably, from about 0.1% to about 20%, and more preferably, from about 0.5% to about 10% by weight of the composition.

Examples of suitable emollients include C₈₋₃₀ alkyl esters of C₈₋₃₀ carboxylic acids; C₁₋₆ diol monoesters and diesters of C₈₋₃₀ carboxylic acids; monoglycerides, diglycerides, and triglycerides of C₈₋₃₀ carboxylic acids, cholesterol esters of C₈₋₃₀ carboxylic acids, cholesterol, and hydrocarbons. Examples of these materials include diisopropyl adipate, isopropyl myristate, isopropyl palmitate, ethylhexyl palmitate, isodecyl neopentanoate, C₁₂₋₁₅ alcohols benzoates, diethylhexyl maleate, PPG-14 butyl ether, PPG-2 myristyl ether propionate, cetyl ricinoleate, cholesterol stearate, cholesterol isostearate, cholesterol acetate, jojoba oil, cocoa butter, shea butter, lanolin, lanolin esters, mineral oil, petrolatum, and straight and branched C₁₆-C₃₀ hydrocarbons.

Also useful are straight and branched chain fatty C₈-C₃₀ alcohols, for example, stearyl alcohol, isostearyl alcohol, behenyl alcohol, cetyl alcohol, isocetyl alcohol, and mixtures thereof. Examples of other suitable emollients are disclosed in U.S. Pat. No. 4,919,934; which is incorporated herein by reference in its entirety.

Other suitable emollients are various alkoxylated ethers, diethers, esters, diesters, and trimesters. Examples of suitable alkoxylated ethers include PPG-10 butyl ether, PPG-11 butyl ether, PPG-12 butyl ether, PPG-13 butyl ether, PPG-14 butyl ether, PPG-15 butyl ether, PPG-16 butyl ether, PPG-17 butyl ether, PPG-18 butyl ether, PPG-19 butyl ether, PPG-20 butyl ether, PPG-22 butyl ether, PPG-24 butyl ether, PPG-30 butyl ether, PPG-11 stearyl ether, PPG-15 stearyl ether, PPG-10 oleyl ether, PPG-7 lauryl ether, PPG-30 isocetyl ether, PPG-10 glyceryl ether, PPG-15 glyceryl ether, PPG-10 butyleneglycol ether, PPG-15 butylene glycol ether, PPG-27 glyceryl ether, PPG-30 cetyl ether, PPG-28 cetyl ether, PPG-10 cetyl ether, PPG-10 hexylene glycol ether, PPG-15 hexylene glycol ether, PPG-10 1,2,6-hexanetriol ether, PPG-15 1,2,6-hexanetriol ether, and mixtures thereof.

Examples of alkoxylated diethers include PPG-10 1,4-butanediol diether, PPG-12 1,4-butanediol diether, PPG-14 1,4-butanediol diether, PPG-2 butanediol diether, PPG-10 1,6-hexanediol diether, PPG-12 1,6-hexanediol diether, PPG-14 hexanediol diether, PPG-20 hexanediol diether, and mixtures thereof. Preferred are those selected from the group consisting of PPG-10 1,4-butanediol diether, PPG-12 1,4-butanediol diether, PPG-10 1,6-hexandiol diether, and PPG-12 hexanediol diether, and mixtures thereof.

Suitable lipids include C₈-C₂₀ alcohol monosorbitan esters, C₈-C₂₀ alcohol sorbitan diesters, C₈-C₂₀ alcohol sorbitan triesters, C₈-C₂₀ alcohol sucrose monoesters, C₈-C₂₀ alcohol sucrose diesters, C₈-C₂₀ alcohol sucrose triesters, and C₈-C₂₀ fatty alcohol esters of C₂-C₆₂-hydroxy acids. Examples of specific suitable lipids are sorbitan diisostearate, sorbitan dioleate, sorbitan distearate, sorbitan isostearate, sorbitan laurate, sorbitan oleate, sorbitan palmitate, sorbitan sesquioleate, sorbitan esquistearte, sorbitan stearate, sorbitan triiostearte, sorbitan trioleate, orbitan tristeate, sucrose cocoate, sucrodilaurate, sucrose distearate, sucrose laurate, sucrose myristate, sucrose oleate, sucrose palmitate, sucrose ricinoleate, sucrose stearate, sucrose tribehenate, sucrose tristearate, myristyl lactate, stearyl lactate, isostearyl lactate, cetyl lactate, palmityl lactate, cocoyl lactate, and mixtures thereof.

Other suitable emollients include mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearate, sorbitan dilaurate, sorbitan stearate, sorbitan laurate, sucrose laurate, sucrose dilaurate, sodium isostearyl lactylate, lauryl pidolate, sorbitan stearate, stearyl alcohol, cetyl alcohol, behenyl alcohol, PPG-14 butyl ether, PPG-15 stearyl ether, and mixtures thereof.

Emulsifiers

The compositions of the invention may also include various emulsifiers. In the final product compositions of the invention, emulsifiers may be included in the amount of up to about 10%, preferably, in the amount of from about 0.5% to about 5% by weight of the composition. The examples of suitable emulsifiers include stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, polyethyleneglycols, polypropyleneglyocis, and mixtures thereof.

Thickeners

The compositions of the invention may also include various thickeners, such as cross-linked acrylates, nonionic polyacrylamides, xanthan gum, guar gum, gellan gum, and the like; polyalkyl siloxanes, polyaryl siloxanes, and aminosilicones. In the final product compositions of the invention, thickeners may be included in the amount of up to about 10%, preferably, in the amount of from about 0.2% to about 5% by weight of the composition.

The specific examples of the suitable thickening silicon compounds include polydimethylsiloxane, phenylsilicone, polydiethylsiloxane, and polymethylphenylsiloxane. Some of the suitable silicon compounds are described in European Patent Application EP 95,238 and U.S. Pat. No. 4,185,017, which are incorporated herein by reference. The compositions of the invention may also include silicone polymer materials, which provide both style retention and conditioning benefits to the hair. Such materials are described in U.S. Pat. No. 4,902,499, which is incorporated herein by reference.

Hair Conditioning Agents

The compositions of the invention may also include hydrolyzed animal protein hair conditioning agents. Croda Incorporated sells an example of a commercially available material under the trade name Crotein Q®. Other examples include urea, glycerol, and propoxylated glycerols, including those described in U.S. Pat. No. 4,976,953, which is incorporated by reference herein.

Hair Setting Agents

The compositions of the invention may also include hair-setting agents to impart styling benefits upon application to hair. The hair setting polymers may be homopolymers, copolymers, terpolymers, etc. For convenience in describing the polymers hereof, monomeric units present in the polymers may be referred to as the monomers from which they can be derived. The monomers can be ionic (e.g., anionic, cationic, amphoteric, zwitterionic) or nonionic.

Examples of anionic monomers include unsaturated carboxylic acid monomers such as acrylic acid, methacrylic acid, maleic acid, maleic acid half ester, itaconic acid, fumaric acid, and crotonic acid; half esters of an unsaturated polybasic acid anhydride such as succinic anhydride, phthalic anhydride or the like with a hydroxyl group-containing acrylate and/or methacrylate such as hydroxyethyl acrylate and, hydroxyethyl methacrylate, hydroxypropyl acrylate and the like; monomers having a sulfonic acid group such as styrenesulfonic acid, sulfoethyl acrylate and methacrylate, and the like; and monomers having a phosphoric acid group such as acid phosphooxyethyl acrylate and methacrylate, 3-chloro-2-acid phosphooxypropyl acrylate and methacrylate, and the like.

Examples of cationic monomers include monomers derived from acrylic acid or methacrylic acid, and a quaternarized epihalohydrin product of a trialkylamine having 1 to 5 carbon atoms in the alkyl such as (meth) acryloxypropyltrimethylammonium chloride and (meth)acryloxypropyl-triethylammonium bromide; amine derivatives of methacrylic acid or amine derivatives of methacrylamide derived from methacrylic acid or methacrylamide and a dialkylalkanolamine having C₁-C₆ alkyl groups such as dimethylaminoethyl (meth)acrylate, diethylaminoethyl (meth)acrylate, dimethylaminopropyl (meth)acrylate, or dimethylaminopropyl (meth) acrylamide.

Examples of the amphoteric monomers include zwitterionized derivatives of the aforementioned amine derivatives of (meth)acrylic acids or the amine derivatives of (meth)acrylamide such as dimethylaminoethyl (meth)acrylate, dimethylaminopropyl(meth)acrylamide by a halogenated fatty acid salt such as potassium monochloroacetate, sodium monobromopropionate, aminomethylpropanol salt of monochloroacetic acid, triethanolamine salts of monochloroacetic acid and the like; and amine derivatives of (meth)acrylic acid or (meth)acrylamide, as discussed above, modified with propanesultone.

Examples of nonionic monomers are acrylic or methacrylic acid esters of C₁-C₂₄ alcohols, such as methanol, ethanol, 1-propanol, 2-propanol, 1-butanol, 2-methyl-1-propanol, 1-pentanol, 2-pentanol, 3-pentanol, 2-methyl-1-butanol, 1-methyl-1-butanol, 3-methyl-1-butanol, 1-methyl-1-pentanol, 2-methyl-1-pentanol, 3-methyl-1-pentanol, t-butanol, cyclohexanol, 2-ethyl-1-butanol, 3-heptanol, benzyl alcohol, 2-octanol, 6-methyl-1-heptanol, 2-ethyl-1-hexanol, 3,5-dimethyl-1-hexanol, 3,5,5-trimethyl-1-hexanol, 1-decanol, 1-dodecanol, 1-hexadecanol, 1-octadecanol, styrene; chlorostyrene; vinyl esters such as vinyl acetate; vinyl chloride; vinylidene chloride; acrylonitrile; alpha-methylstyrene; t-butylstyrene; butadiene; cyclohexadiene; ethylene; propylene; vinyl toluene; alkoxyalkyl (meth)acrylate, methoxy ethyl (meth)acrylate, butoxyethyl (meth)acrylate; allyl acrylate, allyl methacrylate, cyclohexyl acrylate and methacrylate, oleyl acrylate and methacrylate, benzyl acrylate and methacrylate, tetrahydrofurfuryl acrylate and methacrylate, ethylene glycol di-acrylate and -methacrylate, 1,3-butyleneglycol di-acrylate and -methacrylate, diacetonacrylamide, isobornyl (meth)acrylate, n-butyl methacrylate, isobutyl methacrylate, 2-ethylhexyl methacrylate, methyl methacrylate, t-butylacrylate, t-butylmethacrylate, and mixtures thereof.

Examples of anionic hair styling polymers are copolymers of vinyl acetate and crotonic acid, terpolymers of vinyl acetate, crotonic acid and a vinyl ester of an alpha-branched saturated aliphatic monocarboxylic acid such as vinyl neodecanoate; and copolymers of methyl vinyl ether and maleic anhydride, acrylic copolymers and terpolymers containing acrylic acid or methacrylic acid.

Examples of cationic hair styling polymers are copolymers of amino-functional acrylate monomers such as lower alkylamino alkyl acrylate or methacrylate monomers such as dimethyl aminoethylmethacrylate with compatible monomers such as N-vinylpyrrolidone or alkyl methacrylates such as methyl methacrylate and ethyl methacrylate and alkyl acrylates such as methyl acrylate and butyl acrylate.

Miscellaneous Components

The compositions of the invention may also include a wide range of miscellaneous ingredients. Some suitable miscellaneous ingredients commonly used in the cosmetic and personal care industry are described in The CTFA Cosmetic Ingredient Handbook, (2^{nd} Ed., 1992), which is incorporated by reference herein.

Thus, the compositions of the invention may also include one or more absorbents, anti-acne agents, antiperspirants, anticaking agents, antifoaming agents, antimicrobial agents, antioxidants, antidandruff agents, astringents, binders, buffers, biological additives, buffering agents, bulking agents, chelating agents, chemical additives, coupling agents, conditioners, colorants, cosmetic astringents, cosmetic biocides, denaturants, drug astringents, detergents, dispersants, external analgesics, film formers, foaming agents, fragrance components, humectants, keratolytics, opacifying agents, pH adjusters, preservatives, propellants, proteins, retinoids, reducing agents, sequestrants, skin bleaching agents, skin-conditioning agents (humectants, miscellaneous, and occlusive), skin soothing agents, skin healing agents, softeners, solubilizing agents, lubricants, penetrants, plastisizers, solvents and co-solvents, sunscreening additives, salts, essential oils, and vitamins.

The examples of suitable pH adjusters include sodium hydroxide, triethanoleamine, and aminomethylpropanol, and mixtures thereof. If pH adjusters are present in a final product composition, the amount may vary from about 0.01% to about 5%, preferably, from about 0.1% to about 2% by weight of the composition.

The examples of suitable film formers include glycerin/diethylene glycol myristate copolymer, glycerin/diethylene glycol adipate copolymer, ethyl ester of PVM/MA copolymer, PVP/dimethiconylacrylate/polycarbamyl/polyglycol ester, and mixtures thereof. If the film formers are present in the final product compositions, the amount may vary from about 0.1% to about 15.0% by weight of the composition, preferably, from about 0.1% to about 2.5% by weight of the composition.

The examples of suitable vitamins include tocopherol, tocopherol acetate, retinoic acid, retinol, and retinoids.

The examples of suitable anti-acne medicaments include resorcinol, sulfur, salicylic acid, erythromycin, zinc, and benzoyl peroxide.

The examples of suitable skin bleaching or lightening agents include hydroquinone, and kojic acid. The examples of suitable aesthetic components such as fragrances, pigments, colorings, and the like, include panthenol and derivatives (e.g., ethyl panthenol), aloe vera, pantothenic acid and its derivatives, clove oil, menthol, camphor, eucalyptus oil, eugenol, menthyl lactate, witch hazel distillate, allantoin, bisabolol, and dipotassium glycyrrhizinate.

The ingredients described in U.S. Pat. Nos. 5,693,316, 5,597,555, 5,455,025, and 5,302,377, which are thus incorporated herein by reference, may also be used in the compositions of the invention to the extent such use is commensurate with overall description of the compositions provided herein.

The invention is further illustrated in reference to the following non-limiting examples that follow.

The present invention includes the discovery that certain esters of certain alkoxylated aromatic alcohols and fatty carboxylic acids provide particularly desirable properties in terms of reducing or eliminating the need for cyclomethicones and in particular D₄, D₅, and D₆ from cosmetic and personal care products. However, as clearly disclosed in U.S. Pat. Nos. 6,987,195 and 7,217,424, the entireties of which are hereby incorporated by reference, it is possible to use other esters of alkoxylated fatty alcohols and other fatty carboxylic acids, such as those disclosed in the '195 and '424 patents, and in some instances, this can result in cyclomethicone containing or cyclomethicone-free formulations. Thus, in accordance with another aspect of the invention, there is provided cyclomethicone-free formulations or formulations containing a reduced amount of a cyclomethicone using any ester of an alkoxylated aromatic fatty alcohol esterified with any including, without limitation, those described in the aforementioned U.S. patents.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A personal care or cosmetic product comprising:
a) at least one ester of (1) an alkoxylated aromatic alcohol wherein the aromatic alcohol is a substituted or unsubstituted benzyl alcohol, or a substituted or unsubstituted phenol, bound directly or through a connector, to an alkoxy group of 2 to 7 alkoxy units selected from the group consisting of ethoxy and propoxy units and wherein a majority of the alkoxy units are branched propoxy units and (2) a substituted or unsubstituted, saturated or mono-unsaturated, branched or cyclic fatty carboxylic acid of 5 to 8 carbons, the ester being present in the amount of from about 0.1% to about 99% by weight of the product, and
b) at least one functional ingredient present in the amount of from about 0.1% to 60% by weight of the product, wherein the product includes less than about 1% of a cyclomethicone.

2. The cosmetic or personal care product of claim 1 is selected from the group consisting of sunscreens, sun blocks, antiperspirants, deodorants, and colored cosmetics.

3. The cosmetic or personal care product of claim 1 further comprising a connector which is an ethoxy or propoxy unit.
